(19) United States
(12) Patent Application Publication
(10) Pub. No.: US 2011/0295353 A1
(43) Pub. Date: Dec. 1, 2011

(54) PATENT BODY LUMEN STENT

(75) Inventors: Bronwyn Uber Harris, Nashville, TN (US); Thomas Doyle, Nashville, TN (US)

(73) Assignee: VANDERBILT UNIVERSITY, Nashville, TN (US)

(21) Appl. No.: 13/097,939

(22) Filed: Apr. 29, 2011

Related U.S. Application Data
(60) Provisional application No. 61/329,460, filed on Apr. 29, 2010.

(51) Int. Cl. A61F 2/84; A61F 2/82

(52) U.S. Cl. 623/1.11; 623/1.15; 623/1.36

(57) ABSTRACT

A stent implantable in a patent body lumen. The stent comprises a contiguous wire body having a plurality of sequential reversal coils that, when deployed in the patent lumen, are each configured to conform to the shape of the patent lumen and collectively have sufficient radial strength to prevent substantial narrowing of the patent lumen.

### CROSS-REFERENCE TO RELATED APPLICATIONS

This application claims priority to U.S. Provisional Patent Application No. 61/329,460, filed Apr. 29, 2010. The content of this application is hereby incorporated by reference herein.

### BACKGROUND

1. Field of the Invention

The present invention relates generally to implantable stents, and more specifically, to a patent body lumen stent.

2. Related Art

Patients suffer from a number of different conditions that affect the vessels of their vascular system, biliary ducts, esophagus, urethras, etc. (generally and collectively referred to as “body lumens” herein). Generally, such conditions result in the need to implant a stent to open an obstructed body lumen, or to prevent re-closure of a recently opened lumen.

A variety of different procedures are available to doctors, surgeons or other healthcare professionals (collectively referred to as “practitioners” herein) for the implantation of stents within a patient's body lumen. Such procedures broadly fall into one of two categories: open surgical procedures or minimally invasive procedures. Open surgical procedures involve the cutting of incisions in the skin and tissue to provide the surgeon with direct access to a target lumen site.

In minimally invasive procedures, a practitioner accesses a body lumen through relatively small incision(s) or puncture site(s) in the skin or through an anatomical opening. A minimally invasive procedure typically involves the use of laparoscopic or catheter systems which are inserted into the small incision(s), and which are operated remotely by the practitioner. The practitioner indirectly observes the surgical field through an endoscope separate from, or incorporated in, the laparoscopic or catheter system, or by using an imaging device external to the recipient. Due to the minimally invasive access, the patient's recovery times, and hence hospital stay, if any, is generally significantly shorter than open surgical procedures.

### SUMMARY

In one aspect of the present invention a stent implantable in a patent body lumen is provided. The stent comprises: a contiguous wire body having a plurality of sequential reversal coils that, when deployed in the patent lumen, are each configured to conform to the shape of the patent lumen and collectively have sufficient radial strength to prevent substantial narrowing of the patent lumen.

In another aspect of the present invention a system is provided. The system comprises an implantable patent body lumen stent comprising a contiguous wire body having a plurality of sequential reversal coils that, when deployed in a patent lumen, are each configured to conform to the shape of the patent lumen and collectively have sufficient radial strength to prevent substantial narrowing of the patent lumen; and at least one of a stent delivery and a stent removal tool.

### BRIEF DESCRIPTION OF THE DRAWINGS

Illustrative embodiments of the present invention are described herein with reference to the accompanying drawings, in which:

FIG. 1A is a schematic representation of a healthy infant's heart and surrounding vessels having a closed ductus arteriosus;

FIG. 1B is a schematic representation of the heart and surrounding vessels of an infant having hypoplastic left heart syndrome (HLHS);

FIG. 2A is a side view of a patent lumen stent in accordance with embodiments of the present invention, shown in a vessel having a first diameter;

FIG. 2B is a perspective view of the stent of FIG. 2A;

FIG. 3A is a side view of a patent lumen stent in accordance with embodiments of the present invention, shown in a vessel having a second diameter;

FIG. 3B is a perspective view of the stent of FIG. 2A;

FIG. 4 is a schematic representation of an infant's heart and surrounding vessels having a patent lumen stent positioned in the patent ductus arteriosus;

FIG. 5A is a side view of an alternative patent lumen stent in accordance with embodiments of the present invention, shown in a vessel having a first diameter;

FIG. 5B is a side view of an alternative patent lumen stent in accordance with embodiments of the present invention, shown in a vessel having a first diameter;

FIG. 6A is a perspective view of an anchoring arrangement in accordance with embodiments of the present invention;

FIG. 6B is a perspective view of an anchoring arrangement in accordance with embodiments of the present invention;

FIG. 6C is a perspective view of an anchoring arrangement in accordance with embodiments of the present invention;

FIG. 6D is a perspective view of a patent lumen stent secured in a vessel by the anchoring arrangement of FIG. 6A;

FIG. 7 of an alternative patent lumen stent in accordance with embodiments of the present invention;

FIG. 8A is a side view of a stent removal tool, in accordance with embodiments of the present invention;

FIG. 8B is a front view of a stent removal tool of FIG. 8A;

FIG. 8C is a front view of an alternative stent removal tool, in accordance with embodiments of the present invention;

FIG. 9A is a side view of the stent removal tool of FIGS. 8A and 8B shown during use for removal of a patent body lumen stent from a vessel; and

FIG. 9B is a top view of the stent removal tool of FIGS. 8A and 8B shown during use for removal of a patent body lumen stent from a vessel.

### DETAILED DESCRIPTION

Aspects and embodiments of the present invention are generally directed to a stent implantable in a patent body lumen. As used herein, “patent” pronounced, pāt´'nt, is a medical refers to a structure that is generally open and/or unobstructed, and which is not substantially closed. The patent lumen stent comprises a contiguous wire body having a plurality of sequential reversal coils. In some embodiments, the sequential reversals are successive (i.e. adjacent to one another), while in other embodiments additional coils, referred to herein as intermediate coils, are disposed between the reversal coils. When the stent is deployed in a patent lumen, the reversal coils are each configured to conform to the shape of the patent lumen and have sufficient radial strength to prevent substantial narrowing of the patent lumen.

As previously noted, there are numerous medical conditions that result in the need to implant a stent in a patient. Such medical conditions require a stent designed to assist in opening an obstructed body lumen, or to prevent re-closure of a recently opened lumen. Similarly, there are a number of conventional stents designed for this purpose. Because of the general need to open, or re-open an obstructed vessel, conventional stents have a high degree of radial strength. Additionally, because obstructions are a long term problem, these convention stents are generally designed for permanent implantation and cannot be removed without causing significant damage to the body lumen. For example, one common style of prior art permanent stent utilizes a metal tube that is cut in a pattern so that when it is expanded by an internal balloon it forms something analogous to a mesh, a net, or an interlocking set of struts with holes. One such stent is disclosed in U.S. Pat. No. 6,740,113. Over time tissue will grow over the cross links, interlinks, or junctions in the stent, thereby making it difficult or impossible to remove the stent without removing or severely damaging a segment of the vessel wall. In instances where the stenting is meant to be a temporary procedure before ultimate surgical repair, this damage to the vessel wall from overgrowth of the interlocking stent element complicates the subsequent surgical repair, often necessitating removal of large sections of the vessel and/or neighboring vessels. Alternatively, if a practitioner attempts to deploy a stent that has a simple coiled configuration, similar in design to a common coiled spring, it may take a slanted or canted position when deployed.

However, certain patient's suffer from other conditions that require the maintenance of a patent or open body lumen. As used herein, a patent body lumen is a body lumen that is naturally open, rather than which is opened forcibly through, for example, surgical methods. Exemplary patent body lumens may be located throughout a patient's body and include, but are not limited to, vascular system vessels, the trachea, biliary ducts, esophagus, urethras, etc. For ease of illustration, embodiments of the present invention are described below with reference to the maintenance of a specific patent body lumen, namely a patent ductus arteriosus proximate to an infant's heart. However, it would be appreciated that embodiments of the present invention may be utilized in other patent body lumens.

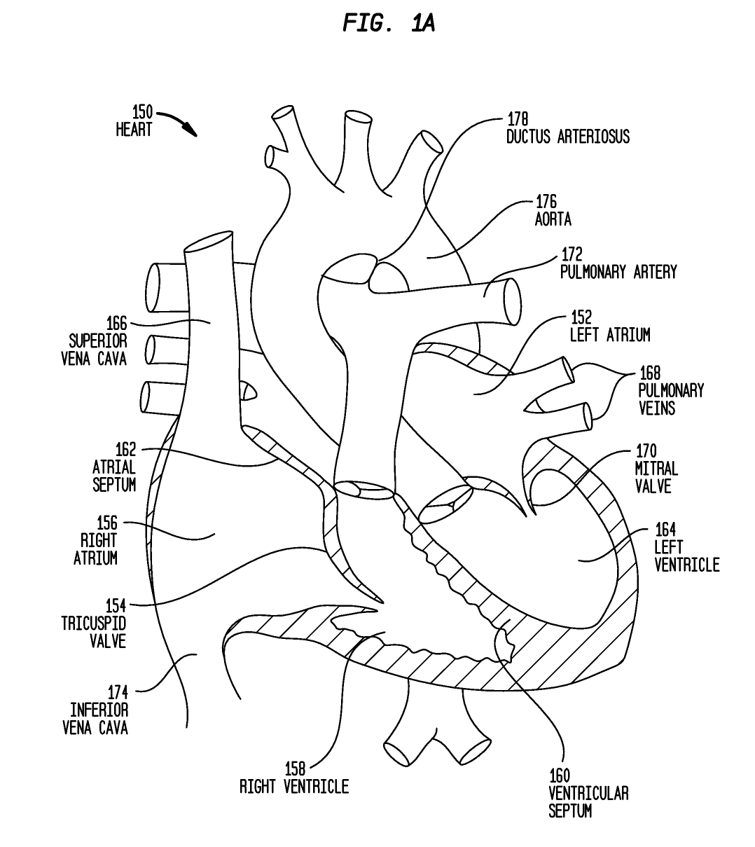

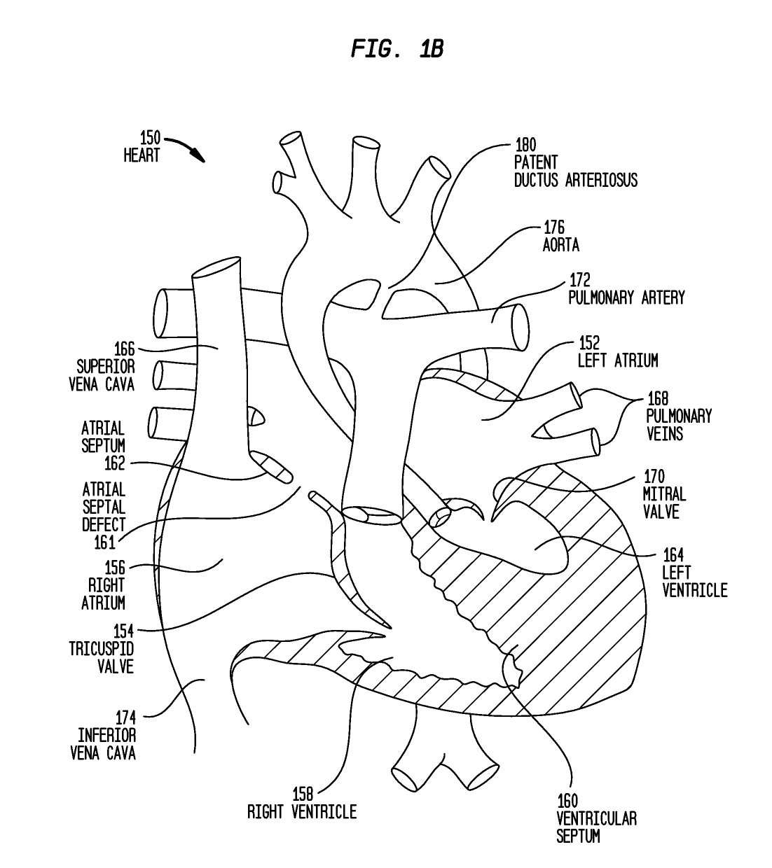

FIG. 1A is a schematic illustration of a healthy infant's heart 150 and surrounding vessels. As shown, heart 150 comprises four separate chambers: the left atrium 152, left ventricle 164, right atrium 156 and right ventricle 158. Ventricles 154 and 158 are separated by the ventricular septum 160, while atria 156 and 152 are separated by atrial septum 162.

In operational, oxygen-poor blood enters into right atrium 156 through two large veins, superior vena cava 166 and inferior vena cava 174. The blood flows from right atrium 156 into right ventricle 158 through an open tricuspid valve 164. Meanwhile, pulmonary veins 168 empty oxygen-rich blood from the lungs (not shown) into left atrium 152. The oxygen rich blood flows from left atrium 152 into left ventricle 154 through open mitral valve 170.

When the ventricles fill with blood, tricuspid valve 164 and mitral valve 210 shut to prevent blood from flowing backward into atria 152 and 156 during ventricular contraction. Ventricular contraction causes oxygen poor blood to flow from right ventricle 158 through pulmonary artery 172 to the lungs. Ventricular contraction also causes oxygen rich blood to leave left ventricle 154 through aorta 176 to the remainder of the body.

When a human infant is born, a blood vessel known as the ductus arteriosus connects the infant's pulmonary artery to the aorta. Typically, within 4 to 10 days after birth, the ductus arteriosus naturally closes. A closed ductus arteriosus 178 is shown in FIG. 1A. The ductus arteriosus closes because of an increase in the arterial oxygen content, along with a decrease in prostaglandin that causes the smooth muscles in the ductus arteriosus to constrict.

In certain circumstances, it is beneficial to maintain a patent (open) ductus arteriosus. For example, a patent ductus arteriosus may be necessary for treatment of certain congenital heart defects such as cyanotic heart defects. Cyanotic heart defects, which account for approximately 25% of all congenital heart defects, are a type of congenital heart defect in which a patient appears blue (cyanotic) due to deoxygenated blood bypassing the lungs and entering the systemic circulation. Typically, the blood bypasses the lungs through shunts between the heart and surrounding vessels, malpositioned vessels, etc. Cyanotic heart defects include, but are not limited to, hypoplastic left heart syndrome (HLHS), Tetralogy of Fallot (ToF), total anomalous pulmonary venous connection, transposition of the great arteries (d-TGA), runcus arteriosus, tricuspid atresia, interrupted aortic arch, coarctation of aorta, pulmonary atresia (PA), and pulmonary stenosis.

FIG. 1B illustrates the heart and surrounding vessels of an infant suffering from HLHS. As noted above with reference to FIG. 1A, oxygen-rich blood returns to left atrium 152 from the lungs, and passes into left ventricle 164, where the blood is pumped out to the body through aorta 176. In HLHS, some or most of the structures on the left side of the heart are small and underdeveloped. FIG. 1B illustrates an infant having an underdeveloped left ventricle 164, mitral valve 170, aorta 176 and aortic valve, as well as an atrial septal defect 161. It would be appreciated that the degree of underdevelopment of such structures differs from infant to infant.

For the patient of FIG. 1B, the maintenance of patent ductus arteriosus 180 is essential. The patent ductus arteriosus provides a pathway to allow oxygenated blood to enter systemic circulation. The patent ductus arteriosus is necessary until the infant is able to grow and become a better candidate for surgical repair.

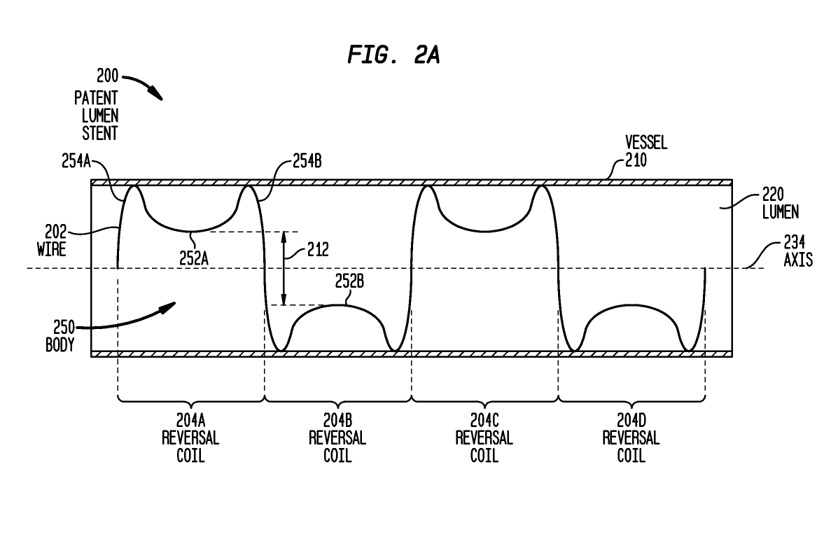

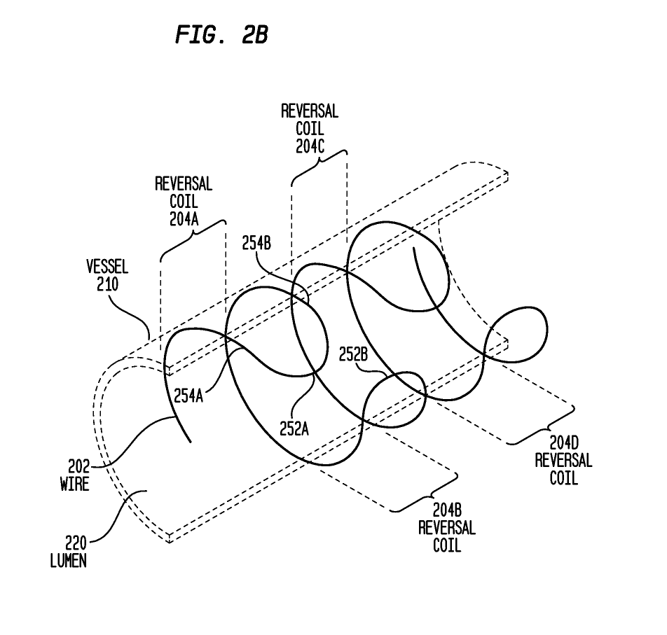

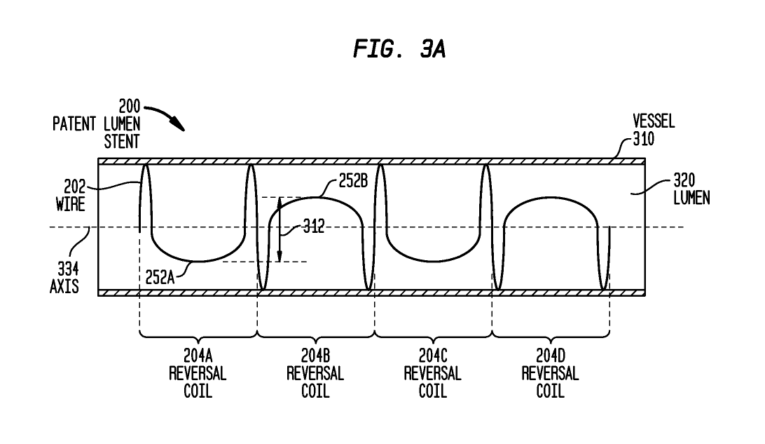

As described in detail below, aspects of the present invention provide a patent lumen stent that, in certain embodiments, is implantable into patent ductus arteriosus 180. When implanted, the patent lumen stent prevents the ductus arteriosus from closing. That is, the stent substantially resists the natural close of patent ductus arteriosus. FIGS. 2A-2B and FIGS. 3A-3B illustrate an exemplary patent lumen stent 200 in accordance with embodiments of the present invention. As detailed below, FIGS. 2A-2B illustrate patent lumen stent 200 positioned within a first exemplary patent body lumen, referred to as vessel 210. FIGS. 3A-3B illustrate patent lumen stent 200 positioned in a second exemplary patent body lumen, referred to as vessel 310.

FIG. 2A is a cross-section of a vessel 210, and provides a side view of patent lumen stent 200 positioned within lumen 220 of the vessel. FIG. 2B is a perspective view of patent lumen stent 200 positioned in lumen 220 of vessel 210. For ease of illustration, vessel 210 is shown using dashed lines in FIG. 2B.

As shown, stent 200 comprises a contiguous wire body 250 formed from, in this exemplary arrangement, a single continuous piece of wire 202. Wire body 250 comprises a plurality of reversal coils 204. As used herein, a reversal coil is a section of wire that begins to form a loop, but which reverses direction at a midpoint 252. In other words, each reversal coil 204 comprises two generally opposing partial loops 254A and 254B which are connected at a midpoint 252. Partial loops 254A and 254B may comprise, for example, quarter loops, half loops, etc. FIGS. 2A and 2B illustrate embodiments comprising four sequential, successive reversal coils 204.

Reversal coils 204 are sufficiently flexible so as to conform to the shape of lumen 220 of vessel 210. However, reversal coils 204 also have sufficient radial strength to prevent closure of lumen 220. That is, reversal coils 204 are configured to substantially resist the natural closure of patent vessel 210. In certain embodiments of the present invention, reversal coils 204 are designed or chosen to have a radial strength that is less than the radial strength of convention stents designed to treat strictures and/or stenosis.

Because patent lumen stent 200 is configured to conform to the shape of lumen 220, the stent may have different configurations depending on the size of vessel in which the stent is implanted. Specifically, the positions of midpoints 252 of sequential reversal coils 204 relative to one another may change depending on the size of the vessel in which stent 200 is implanted. More specifically, in FIG. 2A, an axis 234 is shown. Axis 234 schematically represents a plane that longitudinally bisects vessel 210 to form two substantially symmetrical halves of the vessel. Each half of the bisected vessel has a substantially semi-circular shape. Regardless of the orientation of the bisecting plane, in the configurations of FIGS. 2A and 2B, each reversal loop passes through the bisecting plane one time or, alternatively, will begin and end at the bisecting plane. Additionally, midpoints 252 of successive reversal coils 204 are disposed on opposing sides of the bisecting plane, and are separated from one another by a distance 212.

FIGS. 3A and 3B illustrate an alternative configuration for patent lumen stent 200 positioned in a lumen 320 of a second vessel 310. In this exemplary arrangement, the diameter of lumen 320 is smaller than the diameter of lumen 220 of vessel 210 shown in FIGS. 2A and 2B. As such, reversal coils 204 of stent 200 adopt a more closed configuration. More specifically, similar to FIG. 2A, FIG. 3A includes an axis 334. Axis 334 schematically represents a plane that longitudinally bisects vessel 310 to form two substantially symmetrical halves of the vessel. Each half of the bisected vessel has a substantially semi-circular shape. In the configurations of FIGS. 3A and 3B, each reversal loop passes through the bisecting plane at least two times. In other words, as reversal coils extend about the outer surface of lumen 320, the midpoints 252 will be disposed on an opposing side of the bisecting plane than from the beginning and the end of the reversal coils, thus causing the coil to pass through the bisecting plane at least twice. It would be appreciated that the more closed the configuration of the coils, the more times each coil will pass through a bisecting plane of a vessel.

It would also be appreciated that the reversal coils may have a number of different configurations. For example, in one embodiment the reversal coils may be disposed at different angles relative to each other. In other embodiments, the reversal coils may overlap with each other for added strength, but would not be attached or cross linked with one another. To accomplish this overlap, some of the reversals can assume angles less than a full 180 degrees, for example 135 degrees, 90 degrees, or optionally even less than 90 degrees. Additionally, the reversal sections of the coils may have a keyhole shape enabling the circumferential elements to be closer together, such as closer than twice the radius of curvature of the circular portion of the keyhole shape. Another option is to have the reversal be greater than 180 degrees of reversal. In this case the next deployed segments of radial coils will overlap with the previously deployed coil. The overlapping allows the achievement of increased radial strength or force to resist closer of the patent lumen.

FIGS. 2A-3B are described above with reference to patent lumen stent 200 formed from a single continuous piece of wire 202. It would be appreciated that wire 202 may be any biocompatible material having a substantially linear shape and a suitable cross-section. For example, wire 202 having a circular cross-section, an oval, square, rectangular, or other cross-sectional shape. In one specific embodiment, wire 202 comprises a ribbon shaped wire. In further embodiments wire 202 may comprise a plurality of contiguous elongate elements linearly joined to one another.

In one specific embodiment of the present invention, wire 202 comprises a shape memory material, such as nitnol, having a thickness of approximately 0.014 inches to approximately 0.025 inches. In such embodiments, patent body lumen stent 200 may be formed by coiling wire 202 around an appropriately shaped template and heated in a furnace. This provides stent 200 with an initial shape prior to implantation. In the specific embodiments in which wire 202 is nitnol, the wire and mold may be heated in a furnace for approximately fifteen minutes at approximately 500° C.

It would be appreciated that wire 202 may alternatively be a material that does not have shape memory properties. For example, wire 202 may be stainless steel, tantalum, platinum, platinum iridium, polymers, niobium alloys, cobalt alloys, etc. As would also be appreciated, patent body lumen stent 200 may be manufactured using a number of different processes suitable for each type of material such as, for example, welding, cutting, etching, etc.

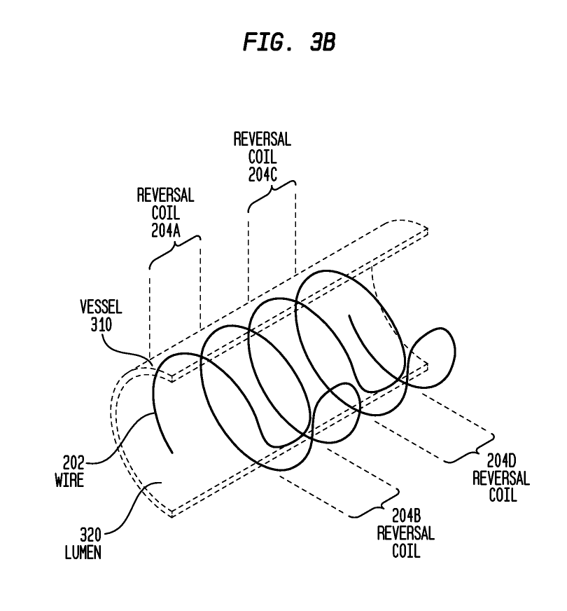

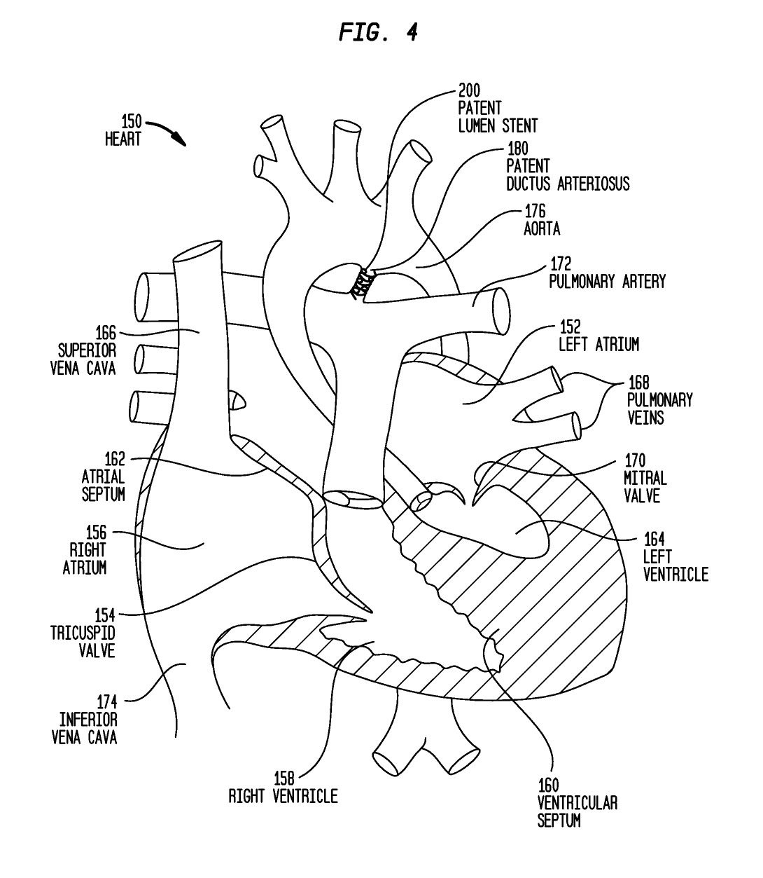

As previously noted, a patent lumen stent in accordance with embodiments of the present invention may be used in numerous different lumens within a patient's body. One such exemplary location detailed above is the patent ductus arteriosus of an infant. FIG. 4 illustrates the infant's heart of FIG. 1B having patent lumen stent 200 implanted in the patent ductus arteriosus 180.

It would be appreciated that patent lumen stent 200 may be implanted into a target patent body lumen, such as patent ductus arteriosus 180, using a number of different techniques. In one exemplary technique, stent 200 is implanted using a percutaneous or minimally invasive approach. In such embodiments, stent 200 is straightened and pulled into a catheter. Such a step may be performed by the practitioner, or may be a manufacturing step.

The distal end of the catheter having the wire therein is guided into aorta 176 or pulmonary artery 172 and through patent ductus arteriosus 180. Once the distal end of the catheter is positioned adjacent the distal opening of patent ductus arteriosus 180, stent 200 is deployed by pushing wire 202 out of the distal end of the catheter, while simultaneously withdrawing the catheter away from distal opening of patent ductus arteriosus 180 toward the proximal opening of patent ductus arteriosus 180.

In certain embodiments, prior to deployment of stent 200, the distal end of the catheter is positioned approximately perpendicular to the opening of patent ductus arteriosus 180. As such, stent 200 exits the distal end of the catheter and deploys at an approximately 90 degree angle relative to the orientation of the catheter. Reversal coils 204 facilitate this angled deployment.

In embodiments of the present invention, it may be necessary to release stent 200 from a feeding wire within the insertion catheter. A number of different techniques known to those skilled in the endoscopic or intravascular interventional device art may be available to allow stent 200 to be disconnected from the feeding wire, including, for example, thermal techniques (dissolving the connection between the feeding wire and stent 200 through melting), or providing a release between the feeding wire and stent 200. The release may be, for example, a screw mechanism, a clamp, etc. In one specific embodiment, the end of the feeding wire encloses a sphere-shaped member disposed at the end of stent 200. The end of the feeding wire is configured to release, and/or reconnect to the sphere-shaped member. In other embodiments, the sphere-shaped member may be replaced with, for example, a loop or hook. Other mechanisms those know to people skilled in the endoscopic or intravascular interventional device art, such as those used in removable vena cava filters, may also be implemented in embodiments of the present invention for retrieval of the released stent.

As noted above, stent 200 may comprise a number of different types of wires. As such, the implantation procedure may differ depending on the materials used for the stent and/or the method of manufacturing the stent.

In some aspects of the present invention, patent lumen stent 200 is implanted in patent ductus arteriosus 180 or other target lumen for a finite period, and is then removed. As mentioned above, conventional stents, such as slit metal tubes, may have a honeycomb and/or cross links that are difficult to remove from a vessel because vessels walls grow over the top of the cross linked segments. As such, a practitioner must remove segments of the aorta and pulmonary artery which makes reconstruction difficult and recovery problematic.

Several features of stent 200 facilitate removal of the stent after the desired period. For example, as shown in FIGS. 2A-3B, stent 200 does not include any structural cross linking between reversal coils 204 that cause tearing of sections of the vessel wall due to ingrowth of the vessel tissue into holes and over the cross links of the stent. Additionally, stent 200 is configured to allow conformance to the shape of the patent lumen, rather than over-expanding and embedding itself hard against the wall of the vessel. This helps can reduce ingrowth and overgrowth of the lumen walls into and onto stent 200. Furthermore, in the exemplary embodiments described above, stent 200 is formed from a contiguous wire, preferably with no permanent interlinks or cross links that is, as described further below, configured to be removed in one piece.

In the embodiments described above, wire 202 is an uncoated wire. In other embodiments of the present invention, stent 200 may be coated with, for example, silicone, polytetrafluoroethylene (PTFE), or a drug-eluting polymer which may reduce ingrowth or overgrowth with the surrounding tissue. In further embodiments, wire 202 may be coated with an anticoagulant, or may include radiopacity enhancements.

As previously noted, FIGS. 2A-4 illustrate a patent lumen stent in accordance with embodiments of the present invention comprising a plurality of sequential, successive reversal coils. FIG. 5A illustrates an alternative patent lumen stent 500 positioned in lumen 520 of a vessel 510 having a plurality of sequential reversal coils 504 that are each separated by an additional coil, referred to herein as intermediate coils 506. That is, successive coils 504 are separated by intermediate coils 506.

As shown in FIG. 5A, stent 500 comprises a contiguous wire body 550 formed from, in this exemplary arrangement, from a single continuous piece of wire 502. Wire body 550 comprises a plurality of sequential reversal coils 504 that are similar to reversal coils 204 described above. Specifically, reversal coils 504 are sufficiently flexible so as to conform to the shape of lumen 520 of vessel 510, but have sufficient radial strength so as to prevent closure of lumen 520.

As noted, stent 500 further comprises a plurality of intermediate coils 506 disposed between adjacent reversal coils 504. Each intermediate coil 506 comprises a turn of wire 502 extending in a single, generally circumferential or circular directional, and which connects two adjacent reversal coils. Similar to reversal coils 504, intermediate coils are configured to conform to the shape of lumen 520, and have sufficient radial strength to resist natural closure of lumen 520. In certain embodiments, the radial strength of intermediate coils 506 may exceed the radial strength of reversal coils 504 and therefore provide greater resistance to vessel collapse.

Furthermore, depending on the desired configuration, vessel diameter, etc., intermediate coils 506 may comprise a full circular turn, a partial circular turn, or greater than a full circular turn. FIG. 5A illustrates embodiments in which intermediate coils 506 comprise approximately a full circular turn.

As noted, FIG. 5A illustrates embodiments of the present invention in which one intermediate coil 506 is disposed between each successive pair of reversal coils 504. FIG. 5B illustrates a patent lumen stent 560 having an alternative arrangement of intermediate coils 506. In these embodiments, at a first side of wire body 552, stent 560 comprises a first reversal coil 504A separated from a second reversal coil 504B by an intermediate coil 506. Similarly, at the second end of wire body 552, stent 560 comprises a third reversal coil 504C separated from a fourth reversal coil 504D by an intermediate coil 506D. However, disposed at the center region of body 552 between successive reversal coils 504B and 504C are two sucessive intermediate coils 506B and 506C. In these embodiments, intermediate coils 506 have a radial strength that exceeds the radial strength of reversal coils 504. As such, the arrangement of FIG. 5B provides increased radial strength for stent 500 in the central region of the stent.

FIGS. 5A and 5B illustrate two exemplary arrangements for a patent lumen stent having intermediate coils disposed between successive intermediate coils. It would be appreciated that other arrangements may be implemented in embodiments of the present invention. For example, in certain embodiments, the arrangement of intermediate coils may be selected to provide for varying levels of radial strength for different types of patent lumens. For example, a patent ductus arteriosus will typically begin to close from the pulmonary artery side of the duct. As such, greater radial strength may be desired or needed on the pulmonary artery side of a stent positioned in a patent ductus arteriosus to prevent such closure. This additional radial strength may be provided by including a greater number of intermediate coils at the pulmonary artery side of the stent, or by having the diameter of thence stiffness or strength of the stent wire be thicker in the region in need of greater strength. In addition, as mentioned above, the angle of a reversal can be angles less than or more than 180 degrees, and can vary from reversal to reversal so that there can be overlap without crosslinking of the intermediate coils or the reversal coils to provide increased radial strength and/or resistance to canting as desired or needed in a particular application and still enable the removability that comes by having a stent with no crosslinking This flexibility could enable the placement of the stent to being away from the opening or end of the lumen. For example the placement could start in the middle, move towards one end, and then after reaching it, have a reversal of more than 180 degrees and then deploy additional coils over the initial coils as deployment proceeds to the other end of the segment which is being reinforced.

As previously noted, a patent lumen stent in accordance with embodiments of the present invention conforms to the shape of a vessel lumen and does not deeply embed itself against the wall of the lumen. As such, in certain embodiments it is possible for the patent lumen stent to migrate after implantation. Such migration may have severe consequences such as life threatening blocking of a vessel or failure to maintain patency of the vessel. To prevent such migration, a patent lumen stent in accordance with embodiments of the present invention may comprise anchoring arrangements disposed at one or both ends of the stent. Such anchoring arrangements are configured to retain the stent in a desired implanted location by securing the stent to body features, such as a vessel wall or opening.

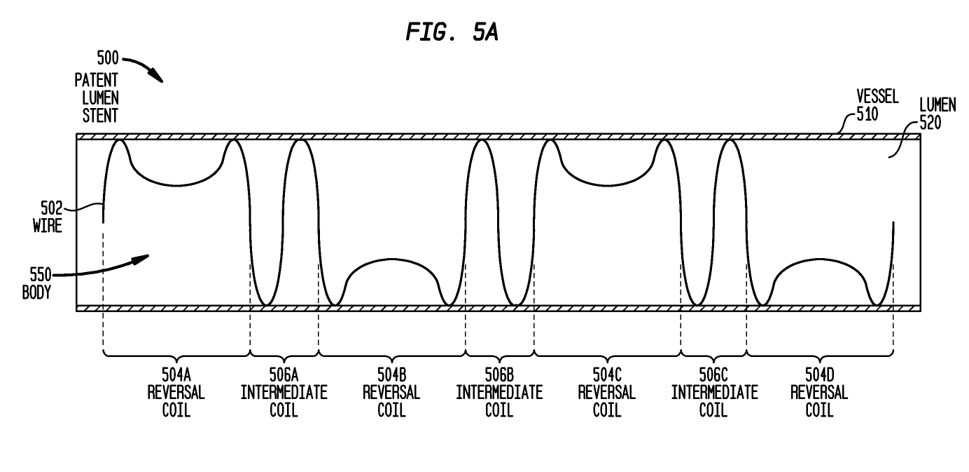

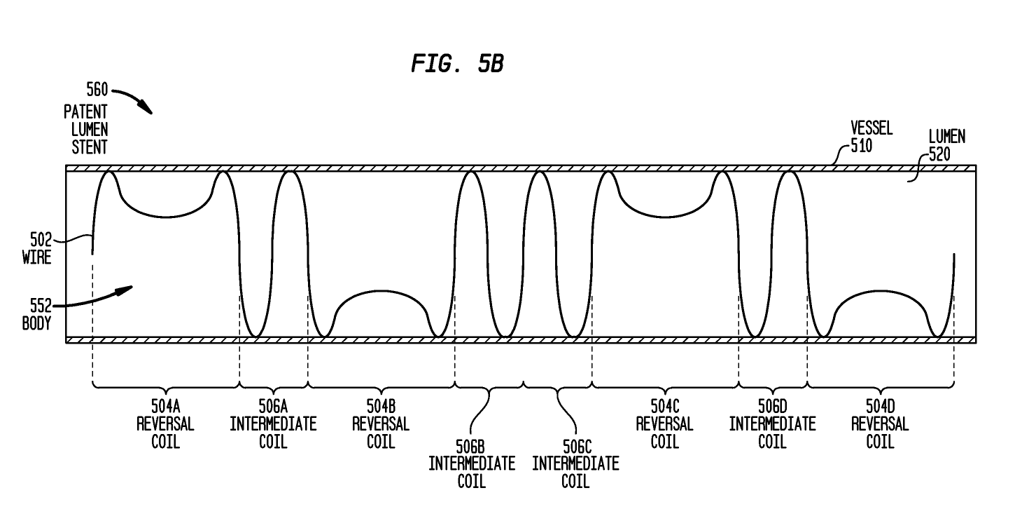

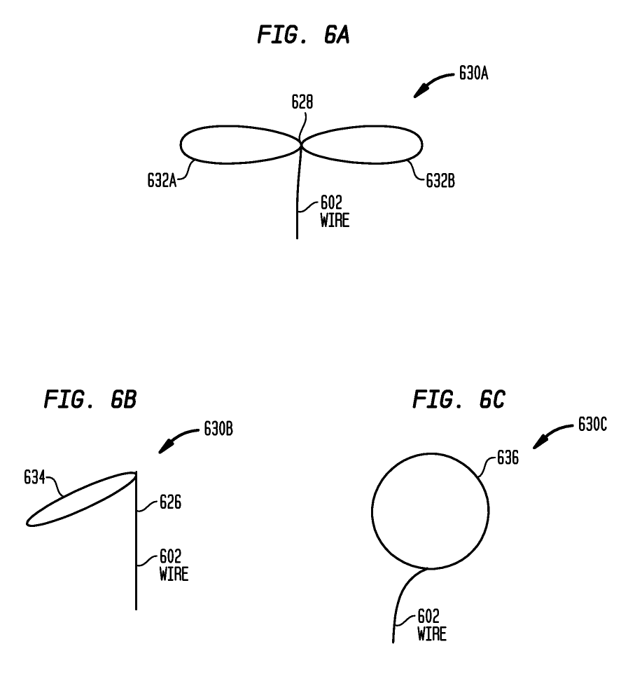

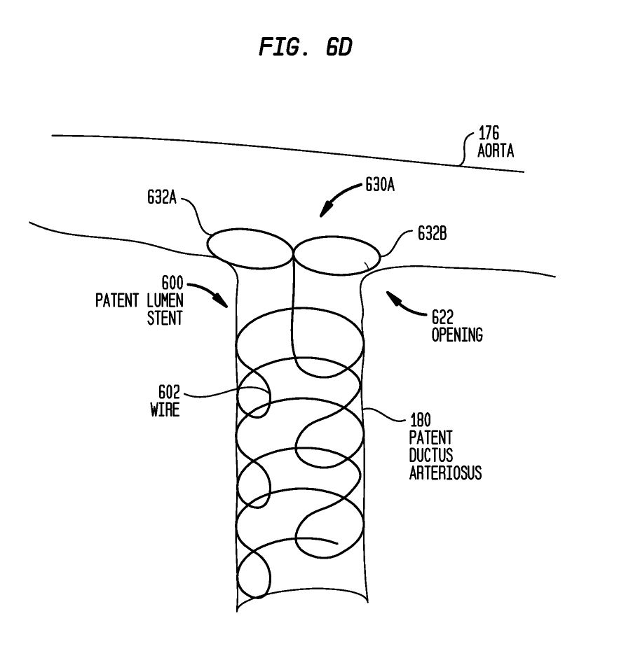

FIG. 6A illustrates an exemplary anchoring arrangement 630A. As shown, anchoring arrangement 630A comprises a section of a wire 602 having a generally lemniscate or figure eight shape. Specifically, anchoring arrangement 630 comprising two co-planar loops 632 overlapping at point 628. In certain embodiments, there is no permanent joint at juncture 628. However, in other embodiments, juncture 628 may be configured to be, when implanted, away from the vessel wall. In such circumstances, because tissue would not be able to grow over the juncture, a permanent joint may be used. One benefit to having one of the loops 632A and 632B permanently joined at juncture 628, at one end of the stent is that the loops may be used as a snare to facilitate capture for subsequent withdrawal or removal of the stent when the patient is able to undergo reconstructive surgery. The loop 630A can be contiguous with wire 602 or it can be separate and joined permanently, for example by welding, to wire 602 if a different property or properties such as material, cross section, or thickness is desired for loop 630A.

FIG. 6D is a perspective view of a patent lumen stent 600 comprising anchoring arrangement 630A. As shown, patent lumen stent 600 is implanted in patent ductus arteriosus 180, and anchoring arrangement 630A is positioned at opening 622 between patent ductus arteriosus 180 and aorta 176. Ovals 632 extend across opening 622 and prevent migration of stent 600 through patent ductus arteriosus 180 into pulmonary artery 172 (FIG. 1A). Although FIG. 6D illustrates anchoring arrangement 630A positioned in aorta 176, it would be appreciated that in other embodiments the anchoring arrangement may be positioned in pulmonary artery 172. In such embodiments, anchoring arrangement 630A prevents migration of stent 600 into aorta 176. For ease of illustration, ovals 632 are shown at an angle relative to patent ductus arteriosus 180. It would be appreciated that in certain embodiments, ovals 632 may be substantially parallel to opening 622 so as to provide minimal interference to the flow of blood through aorta 176.

FIG. 6B illustrates another anchoring arrangement 630B. As shown, anchoring arrangement 630B comprises a hook shaped loop configured to attach to, for example, a surface of a vessel, or to a location where two vessels join with one another. Specifically, anchoring arrangement 630B comprises a substantially linear section 626 of wire 602. Attached to linear section 626 is a second section 634 that extends away from the end of linear section at an acute angle. In the embodiment of FIG. 6B, section 634 has an oval shape. However, it would be appreciated that section 634 may comprise, for example, a single linear section that would fix itself by digging into the vessel wall, or a second linear or curved section to prevent the tip of the wire from digging into the vessel wall, or it may have alternative shapes.

FIG. 6C illustrates another anchoring arrangement 630C. As shown, anchoring arrangement 630C comprises a ring 636 of wire 602. In these embodiments, the diameter of ring 636 is greater than the diameter of the body lumen in which the stent is implanted.

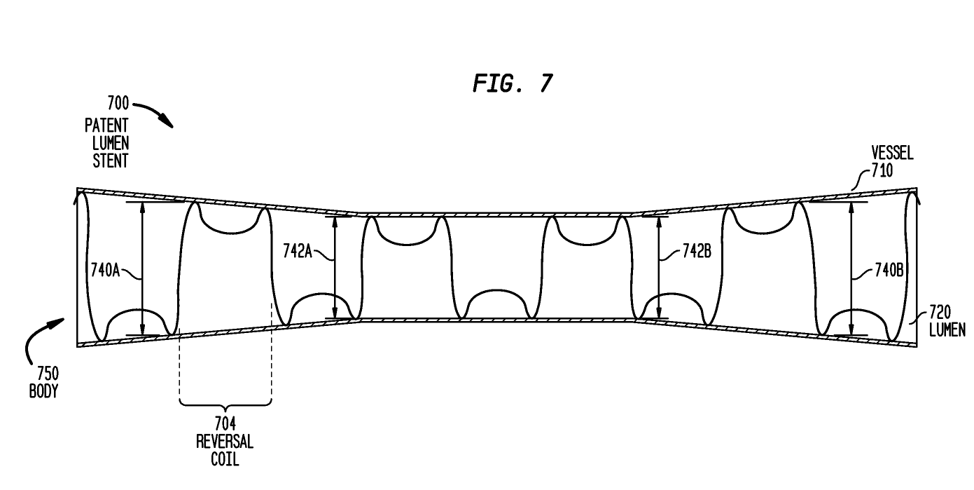

As noted, FIGS. 6A-6C illustrate anchoring arrangements 630 that may be disposed at one or both ends of patent lumen stent. FIG. 7 illustrates an alternative arrangement in which a patent lumen stent 700 is a self-anchoring stent. Similar to the embodiments described above, stent 700 comprises a contiguous body 750 having a plurality of reversal coils 704. For ease of illustration, only one reversal coil 704 is identified.

In the illustrative embodiments of FIG. 7, stent 700 is configured such that the deployed size of the stent is larger at the ends of the stent, and smaller in the middle of the stent. As shown, when deployed, reversal coils 702 expand to have a first diameter 740. This diameter may be the diameter of lumen 720 of vessel 710. However, reversal coils 702 positioned at the center of stent 700 deploy to a second diameter 742 that is smaller than diameter 740. As such, in these embodiments, reversal coils 702 positioned at the center of stent 700 will allow vessel to close further than at the ends, thereby preventing movement of the ends of body 750.

Although the anchoring arrangements of FIGS. 6A-7 may be useful in certain applications, it would be appreciated that anchoring arrangements are not always utilized. For example, it would be appreciated that a patent lumen stent used for tracheomalacia may not utilize an anchor.

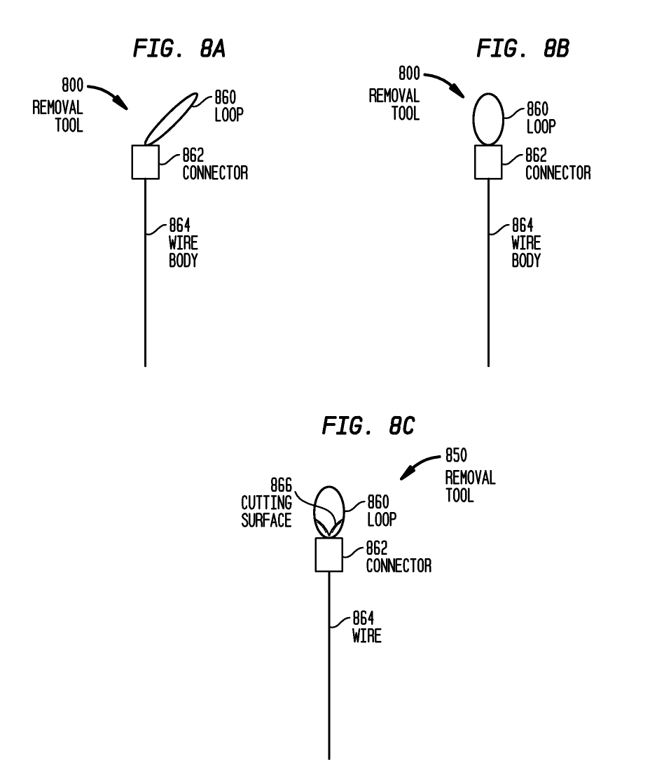

As previously noted, a patent lumen stent in accordance with embodiments of the present invention is implanted in a target lumen for a finite period, and is then removed. In addition, as noted, such removal is facilitated by the features of the patent lumen stent, including the contiguous structure, lack of cross-linking elements, and ability to conform to the shape of the lumen, rather than embed the structure into the lumen walls. Embodiments of the present invention are directed to a tool or device to assist in removing such a patent lumen stent from a target lumen. FIGS. 8A and 8B illustrate side and front views, respectively, of one exemplary stent removal tool 800, while FIG. 8C is a front view of another removal tool 850.

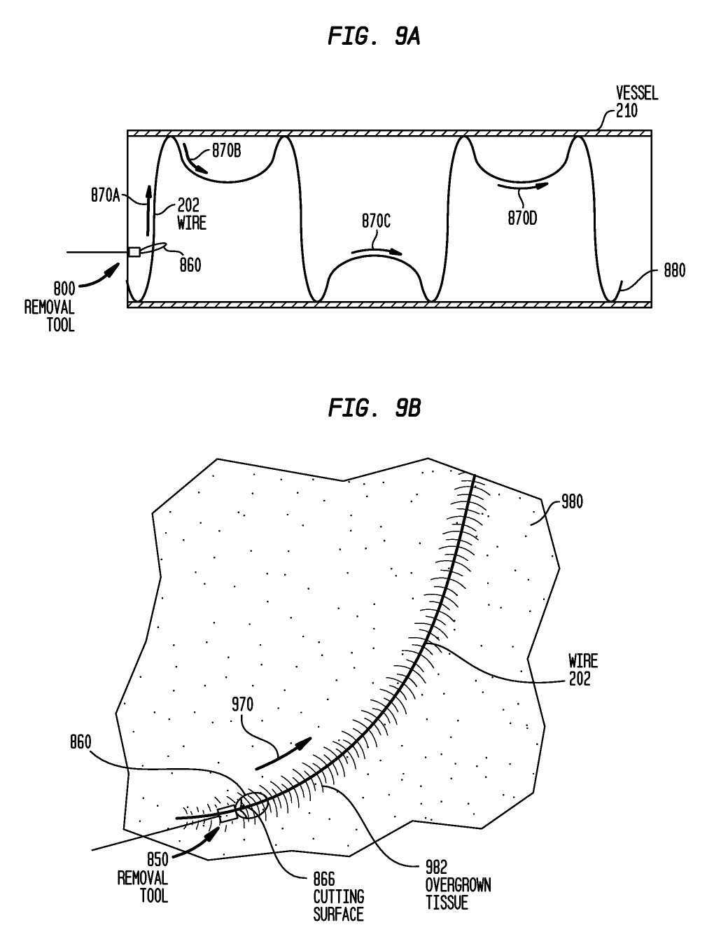

As shown in FIGS. 8A and 8B, removal tool 800 comprises a wire body 864. Disposed at the distal end of wire body 864 is an angled loop 860. Loop 860 is connected to wire body 864 by a connector 862. The use of removal tool 800 will be described below with reference to FIG. 9A.

FIG. 8C illustrates another removal tool 850. Removal tool 850 is substantially similar to removal tool 800 and comprises angled loop 860 connected to wire body 864 by connector. However, as shown in FIG. 8C, removal tool 850 further includes opposing cutting surfaces with loop 860, adjacent to connector 862. As explained below, cutting surfaces 866 may advantageously sever overgrown tissue during the stent removal process.

FIG. 9A is a side view of patent lumen stent 200 described above with reference to FIGS. 2A-4 implanted in vessel 210. To remove stent 200 from vessel 210, a removal tool, such as tool 800 described above, may be advanced from the distal end of a catheter (not shown) positioned in, or adjacent to, vessel 210. As shown, loop 860 is placed over the end of wire 202 so that the wire extends through the loop. Then the free end of the stent is grabbed by a stent retrieval wire using one of the various attachment or retrieval mechanisms known to those skilled in the interventional device art. For example, this could be a ball and clasp, hook and look or a magnetic attachment. As the stent is pulled into the catheter, tool 800 is actuated so that loop 860 is advanced along wire 202, as illustrated by arrows 870, to the end 880 of stent 200. The advancement of loop 860 along wire 202 separates the wire from the vessel walls. In certain embodiments, loop 860 is configured to cut any tissue that has overgrown the stent.

In other embodiments, such as the arrangement shown in FIG. 8C, cutting element(s) may be provided within loop 860 or elsewhere to further facilitate separation of the stent from overgrown tissue. FIG. 9B illustrates the exemplary use of removal tool 850 having cutting surfaces 866 within loop 860. As shown in FIG. 9B, as loop 860 is advanced in along wire 202, as illustrated by arrow 970, cutting surfaces 866 cut through overgrown tissue 982 surrounding wire 202. The cutting of this tissue, coupled with the separation of wire 202 from vessel wall 980 by loop 860, allows removal of stent 200 from the patient without significant tearing or other damage to the patient's vessel.

Additionally, because stent 200 follows a single contiguous path the force required to remove overgrown tissue 982 is significantly less than would be required with a cross-linked structure. This results in less damage than with a cross-linked structure because the single path of the wire results in only one line of tissue being broken at any point, with minimal or no tissue being removed. In contrast, a cross-linked stent will tend to pull a patch of the inner tissue at each cross-link, resulting in significant damage to the wall. Furthermore, in embodiments of the present invention in which portions of the wire overlap and the stent is removed from the end that was last inserted, the overlapping portions are not joined together and will be more easily removed than a cross-link, because the inner or top segment is being removed before the outer or bottom segment.

FIGS. 8A-9B illustrate several exemplary percutaneous removal tools. In an alternative embodiment, a catheter with a side or end hole for receiving the wire stent, for example a longitudinal slit or V-shaped notched at the end of the catheter. As the wire stent is pulled through the hole, the slit or V-shaped notch would contact and place pressure on tissue attached to the wire, thereby separating the wire from the tissue. In certain embodiments, the tip of the catheter with the notch or V-shape may be configured to be free to rotate so it could follow the path of the wire without the entire catheter needing to be rotated.

As noted above, a patent lumen stent in accordance with embodiments of may be used in any patent lumen within a patient's body. As such, although the stent has been primarily described with reference to use in an infant's patent ductus arteriosus, the stent may also be used in a patients trachea to treat tracheomalacia (i.e. a weakness of the trachea making the airway susceptible to collapse), or may be used in the biliary ducts, esophagus, urethras, fallopian tubes, etc.

A significant benefit of the patent lumen stent described herein is that the stent can be made of relatively flexible and thin materials so that deployment is facilitated and action on the lumen is gentler, while the reversals and optional intermediate coils and overlapping of coils increase the resistance to canting or crushing and closure of the lumen that could happen with a simple spirally coiled stent.

While embodiments of the invention have been described with a certain degree of particularity, it is understood that the invention is not limited to the embodiments set forth herein for purposes of exemplification. Modifications and variations of the specific methods and devices described herein will be obvious to those skilled in the art from the foregoing detailed description. Such modifications and variations do not depart from the inventive concept and scope of the present invention and are intended to come within the scope of the appended claims. For example, it would be appreciated that the diameter of wire, amount of wire, proximity of coils, etc., are allow customizable tradeoffs depending on, for example, the desired use of desired configuration.

Reference herein to “one embodiment” or “an embodiment” means that a particular feature, structure, operation, or other characteristic described in connection with the embodiment may be included in at least one implementation of the invention. However, the appearance of the phrase “in one embodiment” or “in an embodiment” in various places in the specification does not necessarily refer to the same embodiment. It is further envisioned that a skilled person could use any or all of the above embodiments in any compatible combination or permutation.

What is claimed is:

1. A stent implantable in a patent body lumen, comprising:
a contiguous wire body having a plurality of sequential reversal coils that, when deployed in the patent lumen, are each configured to conform to the shape of the patent lumen and collectively have sufficient radial strength to prevent substantial narrowing of the patent lumen.

2. The stent of claim 1, wherein the wire body further comprises:
at least one intermediate coil disposed between a pair of sequential reversal coils.

3. The stent of claim 3, wherein the wire body further comprises:
a plurality of intermediate coils disposed between a pair of sequential reversal coils.

4. The stent of claim 1, wherein the wire body further comprises:
one or more intermediate coils disposed between each pair sequential reversal coils.

5. The stent of claim 1, further comprising:
an anchoring arrangement disposed on at least one end of the wire body.

6. The stent of claim 5, wherein the anchoring arrangement comprises:
a portion of the wire body having a lemniscates shape.

7. The stent of claim 5, wherein the anchoring arrangement comprises:
a portion of the wire body forming a hook shaped member.

8. The stent of claim 5, wherein the anchoring arrangement comprises:
a portion of the wire body forming a circle, wherein the diameter of the circle is substantially larger than the diameter of the patent body lumen.

9. The stent of claim 1, wherein the stent comprises a self-anchoring stent having at least one reversal coil configured to have a first height when deployed in the lumen, and at least one reversal coil configured to have a second height when deployed in the lumen.

10. The stent of claim 1, wherein the contiguous wire body comprises a single wire.

11. The stent of claim 1, wherein the contiguous wire body comprises a plurality of joined elongate elements.

12. The stent of claim 1, wherein the contiguous wire body comprises a shape memory material.

13. The stent of claim 12, wherein the shape memory material is nitinol.

14. The stent of claim 1, wherein the wire body has a coating disposed thereon.

15. The stent of claim 14, wherein the coating is at least one of: silicone, polytetrafluoroethylene (PTFE), and a drug-eluting polymer.

16. A system comprising:
an implantable patent body lumen stent comprising a contiguous wire body having a plurality of sequential reversal coils that, when deployed in a patent lumen, are each configured to conform to the shape of the patent lumen and collectively have sufficient radial strength to prevent substantial narrowing of the patent lumen; and
at least one of a stent delivery and a stent removal tool.

17. The system of claim 16, wherein the removal tool comprises:
a wire body; and
an angled loop coupled to the wire body by a connector.

18. The system of claim 17, wherein the loop comprises one or more cutting surfaces disposed therein.

19. The system of claim 16, further comprising:
a catheter configured to deliver the stent to the patent body lumen and configured to positioned the removal tool adjacent the stent.

20. The system of claim 16, wherein the wire body of the stent further comprises:
at least one intermediate coil disposed between a pair of sequential reversal coils.

21. The system of claim 16, wherein the wire body of the stent further comprises:
a plurality of intermediate coils disposed between a pair of sequential reversal coils.

22. The system of claim 16, wherein the wire body of the stent further comprises:
one or more intermediate coils disposed between each pair sequential reversal coils.

23. The system of claim 16, further comprising:
an anchoring arrangement disposed on at least one end of the wire body.

24. The system of claim 23, wherein the anchoring arrangement comprises:
a portion of the wire body having a lemniscates shape.

25. The system of claim 23, wherein the anchoring arrangement comprises:
a portion of the wire body forming a hook shaped member.

26. The system of claim 23, wherein the anchoring arrangement comprises:
a portion of the wire body forming a circle, wherein the diameter of the circle is substantially larger than the diameter of the patent body lumen.
